(19) United States
(12) Patent Application Publication
(10) Pub. No.: US 2013/0183384 A1
(43) Pub. Date: Jul. 18, 2013

(54) IMMEDIATE RELEASE MULTI UNIT PELLET SYSTEM

(71) Applicants: Karl Gerhard WAGNER, Biberach an der Riss (DE); Georg BOECK, Laupheim (DE); Guido RADTKE, Ockenheim (DE)

(72) Inventors: Karl Gerhard WAGNER, Biberach an der Riss (DE); Georg BOECK, Laupheim (DE); Guido RADTKE, Ockenheim (DE)

(73) Assignee: BOEHRINGER INGELHEIM INTERNATIONAL GMBH, Ingelheim am Rhein (DE)

(21) Appl. No.: 13/716,328

(22) Filed: Dec. 17, 2012

(30) Foreign Application Priority Data
Dec. 22, 2011 (EP) 111 95 261.0

(51) Int. Cl. A61K 9/20; A61K 9/28; A61K 31/4439

(52) U.S. Cl. CPC A61K 9/2095; A61K 31/4439; A61K 9/28
USPC 424/474; 546/273.4; 514/338; 427/2.14

(57) ABSTRACT

The present invention relates to a multiple unit pellet system (MUPS) in form of a tablet containing a pharmaceutically active ingredient, characterized in that the MUPS is an optionally coated immediate release pharmaceutical dosage form for oral administration.

### 1. FIELD OF THE INVENTION

The present invention relates to oral multi unit pellet systems containing a pharmaceutically active ingredient, preferably dabigatran etexilate of formula (I) or a pharmaceutically acceptable salt thereof, preferably dabigatran etexilate methansulfonate, most preferably polymorph I of dabigatran etexilate methansulfonate, and methods of preparation and administration thereof, providing an immediate release profile.

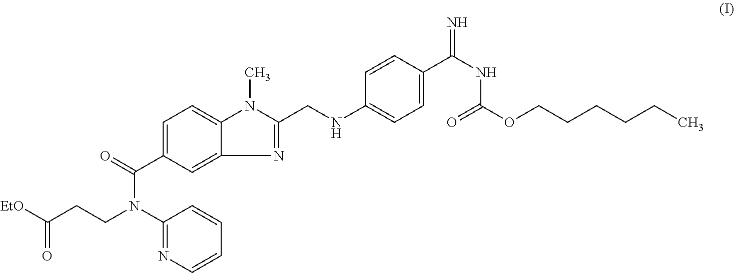 [SMILES: CCCCCCOC(=O)NC(=N)c1ccc(NCc2nc3cc(C(=O)N(CCC(=O)OCC)c4ccccn4)ccc3n2C)cc1]

### 2. BACKGROUND TO THE INVENTION

The compound of formula (I) is known from the prior art and was first disclosed in WO98/37075. It is a potent thrombin inhibitor which can be used for example for the post-operative prevention of deep vein thromboses and in stroke prevention, particularly for preventing strokes in patients with atrial fibrillation. WO 03/074056 discloses the methanesulphonic acid addition salt of dabiagtran-etexilate (ie: dabigatran etexilate methansulphonate) to be particularly useful.

The compound is usually administered orally. In particular, so-called pellet formulations may be used, as disclosed for example in WO 03/074056. According to WO 05/028468 the methansulphonic acid addition salt of dabiagtran etexilate exists in different polymorphic forms. According to WO2009/118322, polymorph I is the preferred polymorph.

Multiple unit pellet systems (MUPS) are seen superior in terms of intra- and inter-individual variability of in vivo drug absorption (Lehmann K, Petereit H U, Dreher D 1993, Pharm Ind 55:940-947) in combination with oral modified release products. These pellets can be filled into hard capsules or be compressed together with suitable fillers and binders into disintegrating pellet-containing tablets. The main focus of the prior art is to retain the modified release properties of the single units, whether those are controlled via a film coat or the embedding of the active ingredient into a polymeric matrix (Abdul S, Chandewar A V, Jaiswal SB 2010., J Control Release 147:2-16). Despite a modified release purpose, seal coated immediate release pellets are formulated in a tablet matrix containing a second API in order to improve stability of the pellets' API (Patel H P, Patel J K, Patel R R 2010, Intl J Pharm Sci 2:448-456).

However, one of the main problems in compressing pellets into disintegrating pellets is damage of the release controlling polymer coatings and/or generation of non disintegrating pellet agglomerates which are formed during tableting and caused by sticky coating polymers or sintering pellets at higher pellets loads (Lopez-Rodriguez F J, Torrado J J, Escamilla C, Cadorniga R, Augsburger L L, Torrado S 1993, Drug Dev Ind Pharm 19:1369-1377). In order to achieve an optimal product the following requirements should be fulfilled (Wagner K G, Krumme M, Schmidt P C 1999. Investigation of the pellet-distribution in single tablets via image analysis. Eur J Pharm Biopharm 47:79-85):
The filler matrix needs to absorb the main fraction of the compression energy and simultaneously provide a acceptable values of tensile strength and hardness.
The fraction of pellets within the formulation should not exceed 60-70% (w/w).
Where coated pellets should be processed into tablets, the polymer coating needs to exhibit a suitable elasticity to cope with some deformation during tableting without rupture.

Based on favorable plastic deformation properties, microcrystalline cellulose (MCC) was proposed as an ideal filler (Bechard S R, Leroux J C 1992, Drug Dev Ind Pharm 18:1927-1944). Used as a filler in fine particle sizes, MCC containing tablets exhibited a higher tensile strength compared to those which were compressed out of pellets and a coarse quality of MCC granules (Alderborn G, Nystrom C 1982. IV, Acta pharm suecica 19:381-390). Simultaneously, MCC types of fine particle sizes act as a stabilizer against segregation in mixtures of larger size- and/or density-gradients (Haubitz H, Mehnert W, Frömming K H 1996. Pharm Ind 58:83-86). Rey described high variances in tablet weight for pellet-containing tablets made from pellets (60 w/w, 850-1700 μm) and fine grade Avicel PH 101, which was attributed to the poor flow properties of the mixture (Rey H 2003. Uniformity of multiunit tablets under pilot plant conditions as a function of unit size and filler composition. Universitöt Tübingen).

The purpose of the present invention is to provide an oral immediate release composition for administration of a therapeutically and/or prophylactically effective amount of a pharmaceutically active ingredient, preferably dabigatran etexilate or a pharmaceutically acceptable salt thereof, most preferably dabigatran etexilate methansulfonate.

It is another purpose of the invention to provide a process that can be used on an industrial scale for preparing immediate release MUPS tablets containing dabigatran etexilate or a pharmaceutically acceptable salt thereof, preferably dabigatran etexilate methansulfonate.

A further aim of the invention is to provide a process which allows the formulation to be manufactured with a reproducible quality.

It is another aim of the invention to provide a manufacturing process which allows the manufacture of a pharmaceutical formulation that contains only one polymorphic form of the active ingredient dabigatran etexilate or a pharmaceutically acceptable salt thereof, preferably dabigatran etexilate methansulfonate.

### 3. DESCRIPTION OF THE FIGURES

FIG. 1 In vitro dissolution of various Dabigatran pellet formulations (110 mg).

FIG. 2a Stress stability of uncoated and coated MUPS tablets: Degradation [%] and dissolution (% dissolved after 10 min, 900 ml 0.01 M HCl, paddle 100 rpm) after 4 weeks at 60° C. in closed twist off brown glass bottles without desiccant.

FIG. 2b Stability of coated MUPS tablets: Degradation [%] and dissolution (% dissolved after 10 min, 900 ml 0.01 M HCl, paddle 100 rpm) after 12 month storage at 25° C./60% rh and 30° C./70% rh in Aluminum Blisters and PP Bottles.

FIG. 3 Structure of dabigatran etexilate pellets.

FIG. 4 Homogeneous distribution of the pellets throughout the MUPS tablet.

FIG. 5 Illustration of process according to the invention.

### 4. DETAILED DESCRIPTION OF THE INVENTION

The present invention achieves the above-stated goals, which relate to a multiple unit pellet system (MUPS) in form of a tablet containing a pharmaceutically active ingredient, wherein the MUPS is an optionally coated immediate release pharmaceutical dosage form for oral administration.

The pharmaceutical compositions according to the invention are intended for oral use and can be used in the dosage form of an uncoated MUPS tablet or a film-coated MUPS tablet.

Preferred, according to the invention, are MUPS tablets comprising a therapeutically and/or prophylactically effective amount of dabigatran etexilate or a pharmaceutically acceptable salt thereof, preferably methansulfonate, more preferably polymorph I, polymorph II or a mixture of Polymorph I and II of dabigatran etexilate methansulfonate, most preferred polymorph I of dabigatran etexilate methansulfonate. Preferably MUPS tablets according to the invention are uncoated. More preferably MUPS tablets according to the invention are having a tablet weight of 100 to 1000 mg, preferably 300 to 900 mg, most prefeably 360 to 800 mg and a loss on drying of below 3.5% (w/w), preferably below 3.0%, most preferably below 2.5

Another object of the present invention is a process for preparing a pharmaceutical composition for oral administration containing an active substance with pH-dependent solubility characteristics and a dose number of more than 1 at pH>5 or one of the pharmaceutically acceptable salts thereof, comprising the steps of:

a) mixing pellets containing a pharmaceutically active substance, preferably dabigatran etexilate or a pharmaceutically acceptable salt thereof, more preferably methansulfonate, most preferably polymorph I of dabigatran etexilate methansulfonate, with excipients selected from the group consisting of one or more fillers, one or more lubricants, one ore more disintegrants and optionally one or more glidants, and
b) compressing the mixture obtained in step a) into a tablet.

Preferable is a process according to the invention, wherein the pellets used in step a) are obtainable by a process as described in US 2005/0095293 or WO2009118322 comprising the steps of:

i) synthesising the core material from one or more pharmaceutically acceptable organic acid(s) with a water solubility of more than 1 g/250 ml at 20° C., optionally with the addition of binders or other technological adjuvants, by pan methods, pelleting plates or by extrusion or spheronisation,
ii) applying an insulating layer consisting of one or more water-soluble, pharmaceutically acceptable polymers, optionally with the addition of plasticisers, separating agents and/or pigments, to the core material,
iii) applying the active substance from a dispersion containing binder and optionally separating agent, and simultaneously or subsequently drying to eliminate the dispersing agent, and
iv) optionally applying a coating of film-forming agents, plasticisers and optionally pigments.

Also preferable is a process according to the invention, wherein the pellets used in step a) or a2) are containing dabigatran etexilate or a pharmaceutically acceptable salt thereof, more preferably methansulfonate, most preferably polymorph I of dabigatran etexilate methansulfonate, with a bioavailability of which is substantially independent of the gastric pH, for oral administration synthesised in each case as described in US 2005/0095293 from

A) a core material,
B) an insulating layer,
C) an active substance layer and
D) an optional coating,
wherein the core material consists of one or more pharmaceutically acceptable organic acid(s) with a water solubility of more than 1 g/250 ml at 20° C., optionally with the addition of binders or other technological adjuvants.

Particularly preferred is a process according to the invention, wherein the mixing step a) comprises

a1) mixing the different fillers and optionally glidants,
a2) mixing the blend of step a1) with pellets containing the active substance, preferably dabigatran etexilate or a pharmaceutically acceptable salt thereof, more preferably methansulfonate, most preferably polymorph I of dabigatran etexilate methansulfonate, and
a3) mixing the blend of step a2) with one or more lubricants.

Further preferred is a process according to the invention, wherein a coating step c) of the MUPS tablet is added comprising the steps

c1) preparation of a coating solution
c2) preheating of the tablet cores in the coater.
c3) spraying of the coating solution onto the surface of the tablet cores within the coater.
c4) drying of the coated tablets.

Further preferred is a process according to the invention, wherein the fillers of step a) are pre-dried.

Particularly preferred is a process according to the invention, wherein the fillers used in step a1) are predried at a temperature range of 100 to 50 C°, preferably 80 to 60 C°, particularly preferred 75 to 65 C°.

Further preferred is a process according to the invention, wherein the fillers of step a) are selected from the group consisting of MCC, microfine cellulose, spray dried lactose MH, alpha-lactose MH, β-lactose AH, compressible sugar, starch, pregelatinized starch, dibasic calcium phosphate, tribasic calcium phosphate, calcium sulfate, mannitol, sorbitol, xylitol, isomaltose, ludipress, pharmatose DCL 40, cellactose, starlac and emdex, preferably cellactose, ludipress α-lactose and mannitol, more preferably starch, microfine cellulose, spray dried lactose and spray dried mannitol, particularly preferred MCC.

Further preferred is a process according to the invention, wherein the glidants of step a) are selected from the group consisting of colloidal silicon dioxide, starch and talc, preferably starch, more preferably talc, particularly preferred colloidal silicon dioxide.

Further preferred is a process according to the invention, wherein the lubricants of step a) are selected from the group consisting of calcium stearate hydrogenated, saccharose fatty acid esters, vegetable oils, vegetable oils, mineral oil, polyethylene glycols, stearic acid, sodium stearyl fumarat, preferably polyethylene glycols and saccharose fatty acid esters, more preferably stearic acid and.sodium stearly fumarate, particularly preferred magnesium stearate.

A further object of the invention are MUPS tablets obtainable by a process according to the invention.

The MUPS tablets can be of any size and shape, preferably the MUPS tablets can be of sizes from 21.0×10.0×9.0 to 11.0×5.0×3.0 mm, preferably from 21.0×10.0×9.0 to 14.0×6.0×4.0 mm, most preferred from 21.0×10.0×8.0 mm to 15.0×7.0×4.0 mm.

Preferably in one MUPS tablet, the amount of active ingredient, preferably of dabigatran etexilate or dabigatran etexilate in form of a pharmaceutically acceptable salt thereof, contained in the pellets can be from 75 to 150 mg, preferably from 110 to 150 mg, and may preferably be sufficient to provide a daily dose administered twice daily at one time.

Further preferable the process of steps a) and b) according to the invention are independently from each other carried out at a relative humidity (r.h.) between 0 and 20%.

The present invention is further directed to the MUPS tablets according to the present invention for use as a medicament. The present invention is further directed to the MUPS tablets according to the present invention for the treatment of the post operative prevention of deep vein thromboses and in stroke prevention, particularly for preventing stroke in patients with atrial fibrillation.

The advantages of the present invention are multifold, e.g.:

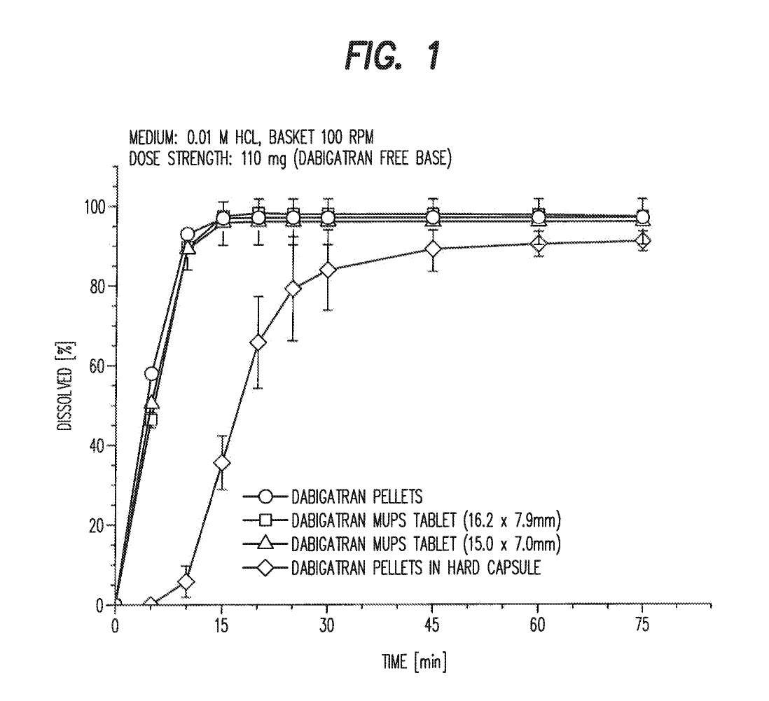

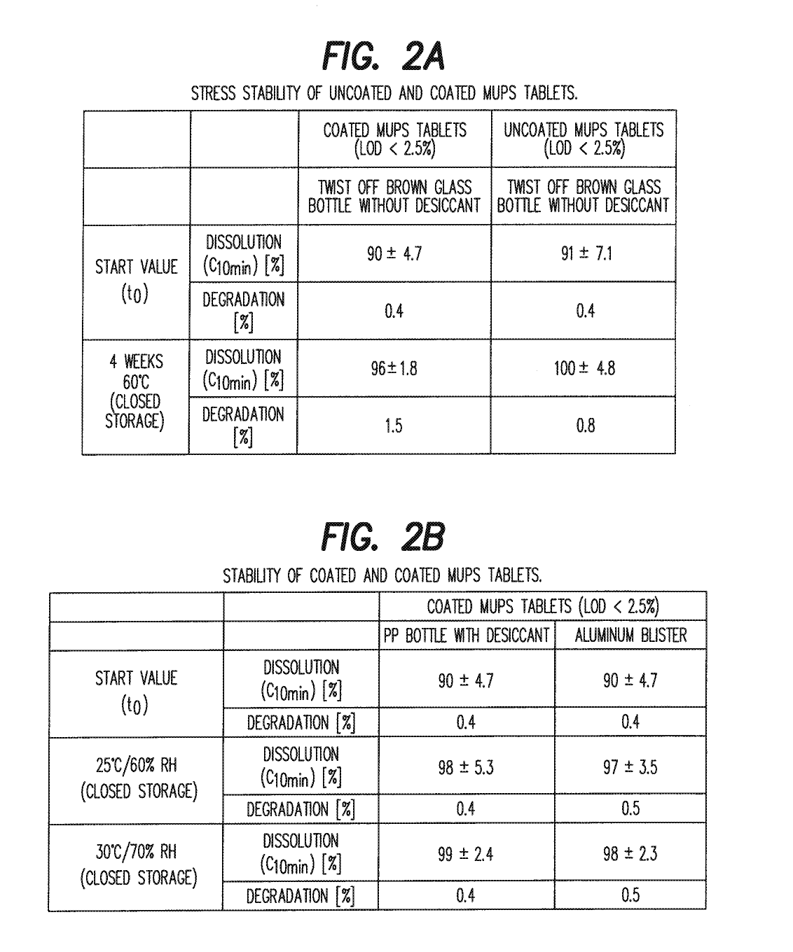

The immediate release MUPS tablets according to the invention exhibit a pellet like dissolution profile. There is no lack time of disintegration compared to pellets in a capsule (FIG. 1).
In spite of the humidity sensitive pellets moisture and stability requirements for the MUPS tablets are met (FIGS. 2a and 2b).
The immediate release MUPS tablets according to the invention exhibit a homogeneous distribution of the pellets throughout the tablet (FIG. 4)

### 5. USED TERMS AND DEFINITIONS

Terms not specifically defined herein should be given the meanings that would be given to them by one of skill in the art in light of the disclosure and the context. As used in the specification, however, unless specified to the contrary, the following terms and abbreviations have the meaning indicated:

### Abbreviations:

CU Content uniformity
HPMC Hypromellose
HPC Hydroxypropyl cellulose
MCC Microcristalline cellulose
MUPS Multiple unit pellet system
TA Tartaric acid
PP Polypropylene

The expression “layer” should be understood in its broadest sense also including a coating or a film or any kind of (partly or fully) surrounding material used in the pharmaceutical sector and having a defined thickness.

The term “pharmaceutically acceptable salt” as used hereinbefore or hereinafter is preferably directed to physiologically acceptable salts with inorganic or organic acids, particularly preferred selected from the group consisting of hydrochloric acid, hydrobromic acid, sulphuric acid, phosphoric acid, fumaric acid, succinic acid, lactic acid, citric acid, tartaric acid, maleic acid and methansulfonic acid, most preferred methansulfonic acid.

Polymorph I of dabigatran etexilate methansulphonate characterized by a melting point of T_{m.p.}=180±3° C. is preferred.

Polymorph II of dabigatran etexilate methansulphonate is characterized by a melting point of T_{m.p.}=190±3° C.

The term “in vitro dissolution” as used hereinbefore or hereinafter is directed to a release characteristic as obtained in a kind of normally used liquid medium for in vitro experiments wherein the release of active ingredient from the immediate release formulation can occur, i.e. for example in in vitro dissolution media, but also in body fluids or simulated body fluids, more in particular in the gastro-intestinal fluids.

In the frame of the present invention the term “immediate” release should mean that the formulation does release the full dose of the active ingredient immediately after oral dosing dependent or independent from the pH value. Preferably at least 85% of the dose will dissolve within 15 min. Thus, similar performance of the MUPS tablet compared to an oral solution must be concluded.

A release characteristic which is pH-independent indicates that the release characteristic is virtually the same in different pH media.

The pH-dependent solubility characteristics of the active substance may mean that, depending on the dose, when administered orally in solid preparations of conventional composition, the active substance is only totally dissolved in the patient's stomach if the liquid present in the stomach has a low enough pH. If the pH in the stomach is elevated (this may be the result of normal physiological variability, illness or co-medication with pharmaceutical compositions that raise the gastric pH), the active substance may not dissolve totally. The effect of the dose of the active substance on its bioavailability can be quantitatively described by means of the concept of the (dimensionless) dose number (Do). The dose number is defined as:

Do=(mo/vo)/cs,

where
Mo=dose (mg),
Vo=liquid volume present (ml) and
Cs=saturation solubility (mg/ml).

According to conventional assumptions, the liquid volume in the stomach after taking a preparation is about 250 ml. (Löbenberg, R., Amidon, G. L.: Modem bioavailability, bioequivalence and biopharmaceutics classification system. New scientific approaches to international regulatory standards (Eur. J. Pharm. Biopharm. 50 (2000) 3-12). At dosages which give a dose number of less than 1, no solubility problems occur. Only if the critical dose number of 1 is exceeded may there be significant reductions in solubility and hence a decreased bioavailability. As a rule the actual problem area only begins at doses which give a dose number significantly above 1, as at least some of the dissolved substance is constantly eliminated from the equilibrium by the absorption process. The active substances contained in the oral formulation according to the invention have a value of less than 1 for the dose number, based on the solubility at pH<2 (i.e. a sufficiently acidic stomach) and a value significantly above 1 for the dose number based on the solubility at pH>5 (i.e. no or vanishingly low gastric acid), i.e. for the oral formulation according to the invention both the degree of pHdependence of the solubility of the active substance and the size of the dose of active substance are of interest.

### 6. PREFERRED EMBODIMENTS

The use of the excipients and ranges specified in Table 1 is preferred in the process steps described herein:

TABLE 1
Preferred excipients and ranges [weight % of uncoated MUPS tablet]

| | | | Range [%] | |
|---|---|---|---|---|
| | | 1 = particularly | (particularly | |
| | | preferred excipient | preferred range, | |
| | | 2 = more preferred | mostly preferred | |
| Excipient class | Name | 3 = preferred | range) | |
| Filler | MCC (d50 ≈ 20 μm) | 1 | 10-70 | |
| | e.g. Avicel PH 105 | | (10-35, 10-25) | |
| Filler | MCC (d50 ≈ 50 μm) | 1 | 10-70 | |
| | e.g. Avicel PH 101 | | (10-35, 10-25) | |
| Filler | MCC (d50 ≈ 90 μm) | 1 | 10-70 | |
| | e.g. Avicel PH 102) | | (10-35, 10-25) | |
| Filler | MCC (d50 ≈ 180 μm) | 1 | 10-70 | |
| | e.g. Avicel PH 200 | | (10-35, 10-25) | |
| Filler | Microfine Cellulose | 2 | 10-70 | |
| | e.g. Elcema G250 | | (10-35, 10-25) | |
| Filler | Spray dried Lactose | 2 | 10-70 | |
| | MH | | (10-35, 10-25) | |
| | e.g. Flowlac | | | |
| Filler | α-Lactose MH | 3 | 10-70 | |
| | e.g. Tablettose | | (10-35, 10-25) | |
| Filler | β-Lactose AH | 3 | 10-70 | |
| | | | (10-35, 10-25) | |
| Filler | Compressible Sugar | 3 | 10-70 | |
| | | | (10-35, 10-25) | |
| Filler | Starch (corn, wheat, | 2 | 10-70 | |
| | rice) | | (10-35, 10-25) | |
| Filler | Pregelatinized starch | 2 | 10-70 | |
| | e.g. Starch 1500 | | (10-35, 10-25) | |
| Filler | Dibasic calcium | 3 | 10-70 | |
| | phosphate | | (10-35, 10-25) | |
| | e.g. Di-Tab, Em- | | | |
| | compress | | | |
| Filler | Tribasic calcium | 3 | 10-70 | |
| | phosphate | | (10-35, 10-25) | |
| | e.g. TriTab, Tri- | | | |
| | Cafos | | | |
| Filler | Calcium Sulfate | 3 | 10-70 | |
| | e.g. Compactrol | | (10-35, 10-25) | |
| Filler | Mannitol | 2 | 10-70 | |
| | | | (10-35, 10-25) | |
| Filler | Sorbitol | 2 | 10-70 | |
| | | | (10-35, 10-25) | |
| Filler | Xylitol | 2 | 10-70 | |
| | | | (10-35,10-25) | |
| Filler | Isomaltose | 3 | 10-70 | |
| | e.g. Galenique iQ | | (10-35, 10-25) | |
| Filler | Ludipress | 2 | 10-70 | |
| | | | (10-35,10-25) | |
| Filler | Pharmatose DCL 40 | 3 | 10-70 | |
| | | | (10-35, 10-25) | |
| Filler | Cellactose | 2 | 10-70 | |
| | | | (10-35,10-25) | |
| Filler | Starlac | 3 | 10-70 | |
| | | | (10-35, 10-25) | |
| Filler | Emdex | 3 | 10-70 | |
| | | | (10-35,10-25) | |
| Disintegrant | Starch NF Corn, | 2 | 5-10 | |
| | Wheat, Potato, Rice | | | |
| Disintegrant | Pregelatinized | 2 | 5-20 | |
| | Starch Binder and a | | | |
| | disintegrant | | | |
| | (Starch 1500°) | | | |
| Disintegrant | Croscarmellose | 1 | 2-4 | (3-4) |
| | Sodium NF | | | |
| | Ac-Di-Sol ® | | | |
| Disintegrant | Sodium Starch | 1 | 2-8 | (3-5) |
| | Glycolate NF | | | |
| | Primojel ® Explotab ® | | | |
| Disintegrant | Crospovidone NF | 1 | 2-5 | (2.5-3.5) |
| Lubricant | Calcium Stearate | 2 | 0.2-2 | |
| | Hydrogenated | | | |
| Lubricant | Vegetable Oils | 3 | 2.0-5 | |
| Lubricant | Magnesium Stearate | 1 | 0.2-2 | (0.5-1.5) |
| Lubricant | Mineral Oil | 3 | 1-3 | |
| Lubricant | Polyethylene Glycols | 2 | 2-5 | |
| Lubricant | Stearic Acid | 2 | 1-4 | |
| Lubricant | Sodium Stearyl | 1bb | 0.5-2 | (0.5-1.5) |
| | Fumarate | | | |
| Lubricant | Alkali Stearates | 2 | 0.2-0.5 | |
| Glidant | Colloidal Silicon | 1 | 0.1-0.8 | (0.3-0.7) |
| | Dioxide (Cab-O-Sil ®, | | | |
| | Syloid ®, Aerosil ®) | | | |
| Glidant | Starch | 2 | 0.2-0.3 | |
| Glidant | Talc | 1 | 0.2-2 | (0.2-0.3) |

Among the optional formulating agents that further may be comprised in the MUPS formulation may be mentioned agents such as microcrystalline cellulose, cellulose derivatives, e.g. ethylcellulose, hydroxypropylmethylcellulose, polyvidone, starch, acacia gum, gelatin, seaweed derivatives, e.g. alginic acid, sodium and calcium alginate, cellulose, preferably microcrystalline cellulose and cellulose derivatives, e.g. ethylcellulose, hydroxypropylmethylcellulose, having useful binding and granulating properties.

Typically the film coat of a MUPS tablet according to the invention represents 2-4%, preferably 3% of the composition and comprises a film-forming agent, a plasticizer, a glidant and optionally one or more pigments. An exemplary coat composition preferably comprises hydroxypropylmethyl-cellulose (HPMC), polyethylene glycol (PEG), talc, titanium dioxide and optionally iron oxide.

The plasticizer can preferably be selected from the group consisting of triethylcitrate and triacetin, particularly preferred polyethylene glycol, preferably in the range of 20-50% (related to the dry mass of the coating polymer).

The filmforming agent can preferably be selected from the group consisting of polyvinyl alcohol, polyvinyl alcohol methacrylic acid copolymer-Type C.mixture, and amino methacrylate copolymer, particularly preferred hypromellose, preferably in the range of 1-3% (related to the core weight of the tablet).

The pigments can preferably be selected from the group consisting of talc, titan dioxide and iron oxides, particularly preferred talc, titan dioxide and iron oxides, preferably in the range of 30-100% (related to the dry mass of the coating polymer).

### 7. PREPARATION

### 7.1 Method for Preparing the Pellets Applied in Process Step a)

Pellets applied in process step a) may be prepared as follows:

### 7.1.1 Formulation Principle of Dabigatran Pellets

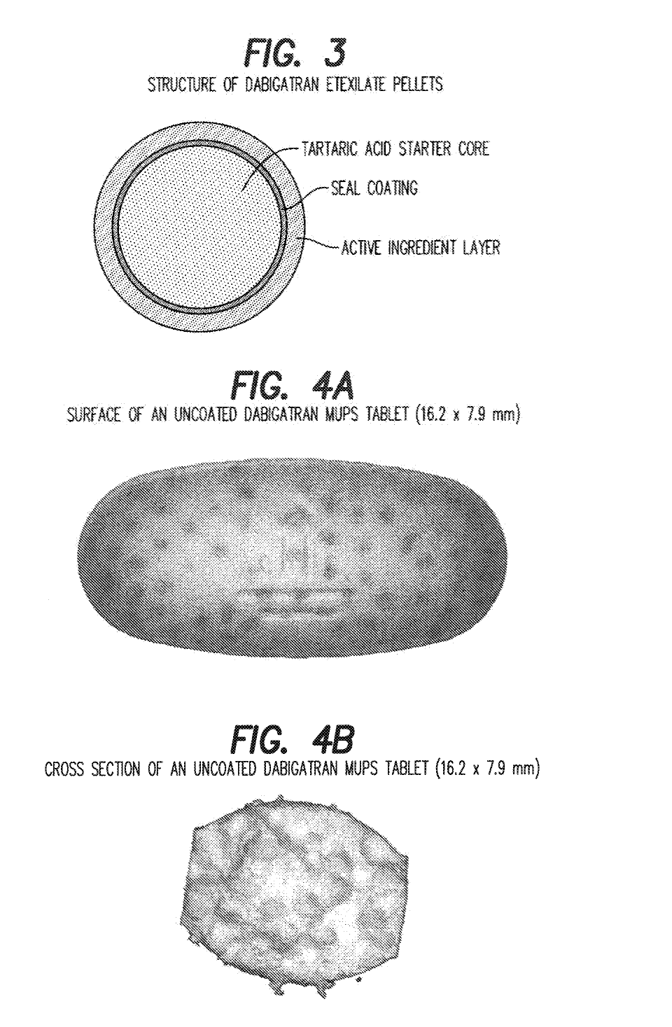

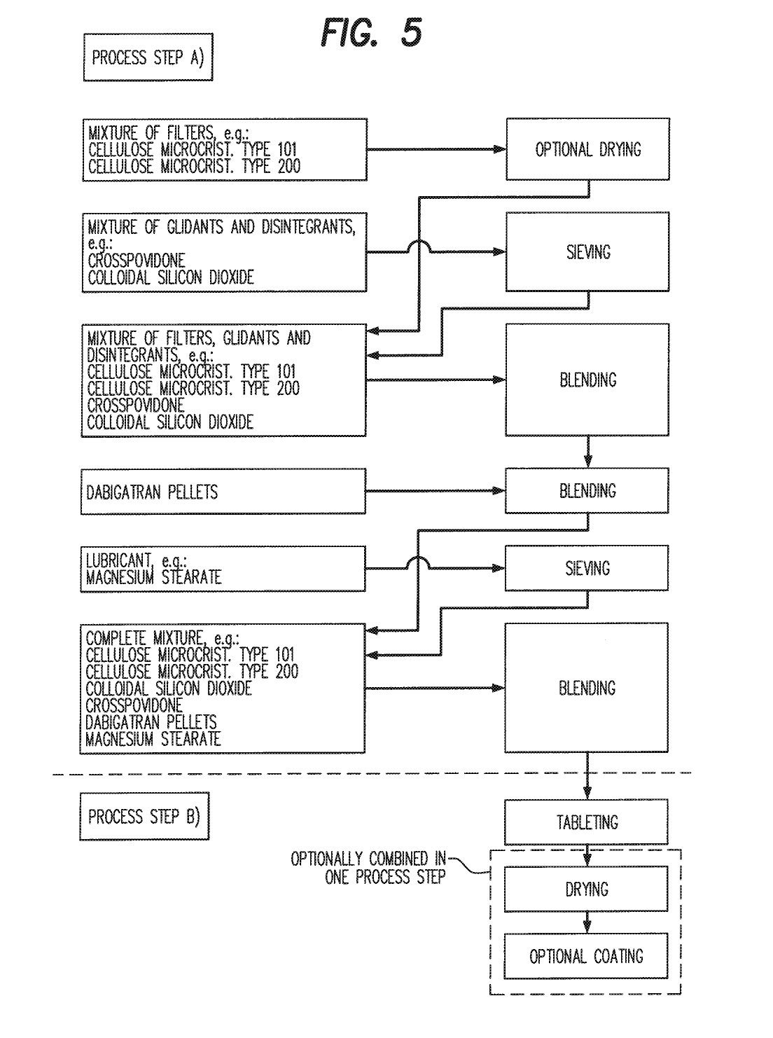

Dabigatran etexilate mesilate is sensitive to hydrolysis especially at low pH. Any formulation containing both organic acid as well as drug substance in close contact is therefore at a very high risk to decomposition, particularly in the presence of humidity. Therefore e.g. tartaric acid and active ingredient are kept separate in the formulation until the moment of application. A multiparticulate pellet approach is chosen. The drug substance is layered from an isopropanolic suspension onto seal coated spherical tartaric acid starter cores with approximately 0.6-0.8 mm diameter (FIG. 3 Structure of dabigatran etexilate pellets'.

The tartaric acid starter cores are isolated with a water soluble barrier film which physically separates the active ingredient from tartaric acid. E.g. hypromellose proved to be appropriate as isolating seal coat, to provide a physical separation of dabigatran etexilate mesilate from the tartaric acid. A detailed description of pellet preparation is described in US 2005/0095293 and WO2009118322.

After ingestion, the MUPS tablets and pellets dissolve and gastric fluids penetrate the drug layer and dissolve the tartaric acid. In the resulting tartaric acid solution the drug substance can dissolve.

### 7.1.2 Manufacturing of the Dabigatran Pellets

The overall manufacturing process of dabigatran etexilate pellets may involve rotating pan coating for layering of the active ingredient pellets (WO 2009/118322 A1). Another manufacturing process involvies a fluid bed process for layering of the active ingredient pellets (WO 2010/007016 A1).

The major operations in the pellet manufacturing process are illustrated in Table 2:

TABLE 2
Pellet manufacturing process

| | |
|---|---|
| Rotating pan process | Fluid bed process |

manufacture of isolated tartaric acid starter pellets
manufacture of active ingredient suspension

| | |
|---|---|
| dabigatran etexilate pellets active | dabigatran etexilate pellets |
| ingredient layering by pan coating | active ingredient fluid bed |
| resulting in dabigatran etexilate | layering resulting in |
| pellets pre-stage (24% drug load) | dabigatran etexilate pellets |
| dabigatran etexilate pellets active | (40% drug load) |
| ingredient layering by pan coating | |
| resulting in dabigatran etexilate | |
| pellets (40% drug load) | |

final blending resulting in dabigatran etexilate pellets

For example according to US 2005/0095293 the core material used is a pharmaceutically acceptable organic acid with a water solubility of >1 g/250 ml at 20° C., such as e.g. tartaric acid, fumaric acid, succinic acid, citric acid, malic acid, glutamic acid and aspartic acid including the hydrates and acid salis thereof, to which a small amount of 1 to 10% by weight, preferably 3 to 6% by weight of a suitable binder is optionally added. The use of a binder may be necessary, for example, if the starting acids are produced by a pan build-up process. If the method used is extrusion or spheronisation, other technological adjuvants such as microcrystalline cellulose will be needed instead of binders. It is also possible to use pure (100%) acid as the starting material if it can be obtained in a sufficiently narrow range of particle sizes. The pharmaceutically acceptable organic acids used are preferably tartaric acid, fumaric acid, succinic acid or citric acid; tartaric acid is particularly preferred. As binder, it is possible to use gum arabic or a partially or totally synthetic polymer selected from among the hydroxypropylcelluloses, hydroxypropyl-methylcelluloses, methylcelluloses, hydroxyethylcelluloses, carboxymethylcelluloses, polyvinylpyrrolidone, the copolymers of N-vinylpyrrolidone and vinyl acetate, or combinations of these polymers; gum arabic is preferred. The spherical core material preferably has an average diameter of 0.4-1.5 mm. The content of the pharmaceutically acceptable organic acid is usually between 30 and 100% in the core material.

To increase the durability of the finished product it is advantageous to coat the core material before the application of the active substance with an insulating layer based an a water-soluble, pharmaceutically acceptable polymer. Examples of such water-soluble polymers include for example gum arabic or a partially or totally synthetic polymer selected from among the hydroxypropylcelluloses, hydroxypropylmethylcelluloses, methylcelluloses, hydroxyethylcelluloses, carboxymethylcelluloses, polyvinylpyrrolidone, the copolymers of N-vinylpyrrolidone and vinyl acetate, or combinations of these polymers. Gum arabic or a hydroxypropylmethylcellulose is preferably used. If desired, the coating with the water-soluble, pharmaceutically acceptable polymer may be carried out with the addition of suitable plasticisers, separating agents and pigments, such as for example triethylcitrate, tributylcitrate, triacetin, polyethyleneglycols (plasticisers), talc, silicic acid (separating agents), titanium dioxide or iron oxide pigments (pigments). The active substance layer contains the active substance as well as binders and optionally separating agents. Suitable binders include for example hydroxypropylcellulose, hydroxypropylmethylcellulose, methylcellulose, hydroxyethylcellulose, carboxymethylcellulose, polyvinylpyrrolidone, copolymers of N-vinylpyrrolidone and vinyl acetate or combinations of these polymers. Preferably, hydroxypropylcellulose or copolymers of N-vinylpyrrolidone and vinyl acetate are used. The addition of separating agents such as e.g. talc, magnesium stearate or silicic acid serves to prevent the particles from aggregating during the process. The preferred active substance content is not more than 60%, preferably not more than 50% of the pharmaceutical composition.

7.2 Method for preparing the MUPS tablets according to process step a) and b)

Glidants (e.g. colloidal silicon dioxide) and disintegrants (e.g. crosspovidone) are sieved (e.g. cone sieve of mesh size 0.8 mm) onto the mixture of fillers (e.g. microscrystalline cellulose type 101 and 200) as a delumping step. The resulting mixture is subsequently blended in a suitable mixer (e.g. tumbling mixer) before the fraction of dabigatran pellets is added followed by a further mixing step. To complete the tableting mixture a suitable lubricant (e.g. magnesium stearate) is sieved onto the obtained blend and further blended (e.g. tumbling blender). The resulting mixture is further processed into tablets using a suitable tablet machine (e.g. rotary tablet press). The obtained tablet cores are dryed (e.g. tray dryer) in order to achieve a loss of drying (LOD) preferably below 2.5% (w/w). Optionally, the drying step might be combined with the coating step (e.g. drum coater) choosing respective inlet air and spraying conditions. Another alternative to obtain tablet cores with a LOD below 2.5% (w/w) is to use pre-dryed fillers (e.g. microcrystalline cellulose) and further processing of the mixture at humidities below 20% rh.

### 8. EXAMPLES

The following examples serve to illustrate the processes carried out by way of example for preparing the MUPS tablets according to the invention. These are to be understood as being an illustration of the invention, without limiting it to its subject-matter.

### Preparation of the Tablet Cores:

The fillers microcristalline cellulose types 101 and 200 are weighed into a stainless steel drum. Disintegrant (crosspovidone) and colloidal silicium dioxide are weighed and passed together through a 0.8 mm sieve onto the mixture of the fillers. The resultung mixture is blended for 10 min at 32 rpm in a tumbling mixer before the respective fraction of dabigatran pellets is added to the mixture and further blended for another 10 min at 32 rpm. Finally, magnesium stearate is passed through a 0.8 mm sieve onto the excipient-pellet mixture and blended for 10 min using the tumbling mixer at 32 rpm.

The resulting tableting mixture is further processed on a rotary tablet press (Fette P1200) at 50.000 to 125.000 tablets per hour. Tablets of 530±10 mg (corresponds to 110 mg dabigatran free base) are compressed using 16.2×7.9 mm oval shaped tooling at a main compression force of 9±1 kN.

Subsequently the tablet cores are dryed for 24 hours at 70° C. in a tray dryer. After drying the tablet cores displayed a loss of drying (105° C., 15 min, Mettler moisture analyzer Hg 63) of below 2.5% (w/w).

Alternatively the fillers microcristalline cellulose types 101 and 200 are predryed for 24 h at 70° C. in a tray dryer and further processed as described above at humidities of the ambient air of below 20% rh.

### Optional Coating of the Tablet Cores:

The coating dispersion is prepared by dissolving hypromellose (Methocel E 5 prem.) in water and subsequently adding polyethylene glycol (Macrogol 6000). After all polyethylene glycol is dissolved and any foam formed during the dissolution process of the excipients restituted all pigments (ioron oxide yellow, red and black together with talc and titan dioxide) were dipersed in the polymer solution using a homogenizer (Ultra Turrax, 5000 rpm 15 min). Coating of 1.5 kg tablet cores are performed in a drum coater (Glatt GMPC I) using a drum of 30 cm in diameter rotating at 15 rpm. The spray nozzle of 1.2 mm opening diameter works at a pressure of 1.0 bar and a spray rate of 12 g/min. The drying air floated through the coating drum at 100 m³/h and 60° C.

### Example 1

One embodiment of the qualitative and quantitative composition of a dabigatran MUPS tablet 110 mg (free active substance base) is shown in Table 3.

TABLE 3

| Ingredients | [mg/tablet] | [%/tablet] |
|---|---|---|
| Dabigatran pellets containg | 317.090 | 59.83 |
| 126.83 ⁽¹⁾ mg dabigatran etexilate | | |
| methansulfonate per tablet | | |
| Cellulose microcristalline | 95.400 | 18.00 |
| (Typ 101 INT) | | |
| Crospovidon | 15.900 | 3.00 |
| Magnesium stearate | 2.650 | 0.50 |
| Microcel MC-200 | 96.310 | 18.17 |
| Colloidal silicon dioxide | 2.650 | 0.50 |
| Total | 530.00 | 100.00 |

⁽¹⁾ corresponds to 110 mg of free active substance base

The dabigatran pellets used in this embodiment were manufactured according to WO 2010/007016 A1. The qualitative and quantitative composition is depicted in Table 4.

TABLE 4

| Ingredient | Amount [mg] per tablet |
|---|---|
| Dabigatran etexilate methansulfonate | 126.83⁽²⁾ |
| Acacia (gum arabic) | 6.50 |
| Tartaric acid | 129.9 |
| Hydroxymethyl-propylcellulose 2910 | 3.27 |
| Dimethylpolysiloxane 350 | 0.06 |
| Talc | 25.16 |
| Hydroxypropylcellulose | 25.37 |
| Total | 317.09 |

⁽²⁾corresponds to 110 mg of free active substance base

### Example 2

One embodiment of the qualitative and quantitative composition of a dabigatran MUPS tablet is shown in Table 5. Dabigatran pellets used in this embodiment are of the same composition as disclosed in example 1 (see Table 4).

TABLE 5

| Ingredients | [mg/tablet] | [%/tablet] |
|---|---|---|
| Dabigatran pellets containg | 317.090 | 59.83 |
| 126.83 ⁽³⁾ mg dabigatran etexilate | | |
| methansulfonate per tablet | | |
| Avicel PH 200 | 95.400 | 18.00 |
| Cellulose Mikrokrist. Typ 101 | 96.310 | 18.17 |
| Crospovidon (Kollidon CL-SF) | 15.900 | 3.00 |
| Magnesium stearate | 2.650 | 0.50 |
| Colloidal silicon dioxide | 2.650 | 0.50 |
| Total tablet | 530.00 | 100.00 |
| Coating | | |
| Ferric oxide yellow 17015 | 0.290 | 2.00 |
| Ferric oxide red 17009 | 0.290 | 2.00 |
| Ferric oxide Sicopharm-black 80 | 0.058 | 0.40 |
| Hypromellose (Methocel E5 Prem) | 7.250 | 50.00 |
| PEG 6000 | 0.725 | 5.00 |
| Talc | 2.900 | 20.00 |
| Titan dioxide | 2.987 | 20.60 |
| Water | 101.5 | — |
| Total | 544.50 | 100.00 |

⁽³⁾ corresponds to 110 mg of free active substance base

### 9. INSTRUMENTS AND ANALYTICAL METHODS

The instruments and conditions listed in Table 6 may be used for the process according to the invention:

TABLE 6
Production equipment and settings

| Process | Equipment | Setting |
|---|---|---|
| Drying | Tray dryer | 70° C., 24 h, |
| Blending | Tumbling mixer (type | 32 rpm; 10 min/step |
| | Röhnrad) | |
| Sieving | Kressner Handsieb | 0.8 mm |
| Tableting | Fette P 1200 | 50.000-125.000 tablets/hour |
| | | Tooling: 16.2 × 7.9 mm |
| | | Compression force: 9 ± 1 kN |
| Dissolving/ | Stirrer | 500 rpm, 10 min. |
| dispersing | | 2000 rpm, 15 min. |
| | Ultra Turrax | 5000 rpm, 2 min. |
| Coating | GMPC I, Glatt | Drum diameter: 30 cm |
| | Coater | Rotating speed, drum: 15 UpM- |
| | | Nozzle: 1.2 mm |
| | | Tube: 2.4 × 1.6 × 2.4 mm |
| | | Spray rate: 12 g/min |
| | | Spraying pressure: 1 bar |
| | | Drying air, temp.: 60° C. |
| | | Drying air, flow: 100 m³/h |

The instruments and conditions listed in Table 7 may be used for the analysis of the invention:

TABLE 7

| Analytical equipment | Process Equipment |
|---|---|
| Assay and degradation | HPLC (HP Series 1100) |
| LOD | Mettler moisture analyzer hg 63 |
| | (105° C., 15 min) |
| Dissolution | Apparatus 2 USP (Sotax AT7) |
| Temperature/moisture measurement | Thermokon WRF02-PT100/Vaisala |
| for determination of r.h. | HMD60U |

We claim:

1: A multiple unit pellet system (MUPS) in form of a tablet containing a pharmaceutically active ingredient, wherein the MUPS is an optionally coated immediate release pharmaceutical dosage form for oral administration.

2: A pharmaceutical dosage form according to claim 1, wherein the MUPS tablet comprises a therapeutically and/or prophylactically effective amount of dabigatran etexilate or a pharmaceutically acceptable salt thereof.

3: The MUPS tablet according to claim 1 or 2 wherein the MUPS tablet is uncoated.

4: The MUPS tablet according to claim 1 or 2 having a tablet weight of 100 mg to 600 mg.

5: A process for preparing a pharmaceutical composition for oral administration containing a pharmaceutically active substance with pH-dependent solubility characteristics and a dose number of more than 1 at pH>5 or one of the pharmaceutically acceptable salts thereof, comprising the steps of:
(a) mixing pellets containing active substance with excipients selected from the group consisting of one or more fillers, one or more lubricants, one ore more disintegrants and optionally one or more glidants, and
(b) compressing the mixture obtained in step (a) into a tablet.

6: The process according to claim 5 characterized in that the active substance is dabigatran etexilate or a pharmaceutically acceptable salt thereof.

7: The process according to claim 6, wherein the pellets used in step (a) are obtained by a process comprising the steps of:
(i) synthesising the core material from one or more pharmaceutically acceptable organic acid(s) with a water solubility of more than 1 g/250 ml at 20° C., optionally with the addition of binders or other technological adjuvants, by pan methods, pelleting plates or by extrusion/spheronisation,
(ii) applying an insulating layer consisting of one or more water-soluble, pharmaceutically acceptable polymers, optionally with the addition of plasticisers, separating agents and/or pigments, to the core material,
(iii) applying the active substance from a dispersion containing binder and optionally separating agent, and simultaneously or subsequently drying to eliminate the dispersing agent, and
(iv) optionally applying a coating of film-forming agents, plasticisers and optionally pigments.

8: The process according to claim 5, wherein the pellets used in step a) contain dabigatran etexilate or a pharmaceutically acceptable salt thereof with a bioavailability of which is substantially independent of the gastric pH, for oral administration synthesised from
A) a core material,
B) an insulating layer,
C) an active substance layer and
D) an optional coating,
wherein the core material consists of one or more pharmaceutically acceptable organic acid(s) with a water solubility of more than 1 g/250 ml at 20° C., optionally with the addition of binders or other technological adjuvants.

9: The process according to claim 5, wherein the mixing step (a) comprises
(a1) mixing the different fillers and optionally glidants,
(a2) mixing the blend of step (a1) with pellets containing the pharmaceutically active substance, and
(a3) mixing the blend of step (a2) with one or more lubricants.

10: The process according to claim 5 further comprising a coating step (c), wherein the coating step comprises:
(c1) preparation of a coating solution,
(c2) preheating of the tablet cores in the coater,
(c3) spraying of the coating solution onto the surface of the tablet cores within the coater, and
(c4) drying of the coated tablets.

11: The process according to claim 5, wherein steps (a) and (b) are independent from each other and carried out at a relative humidity between 0 and 20%.

12: The process according to claim 5, wherein the fillers of step (a) are selected from the group consisting of MCC, microfine cellulose, spray dried lactose MH, alpha-lactose MH, β-lactose AH, compressible sugar, starch, pregelatinized starch, dibasic calcium phosphate, tribasic calcium phosphate, calcium sulfate, mannitol, sorbitol, xylitol, isomaltose, ludipress, pharmatose DCL 40, cellactose, starlac and emdex.

13: The process according to claim 5, wherein the glidants of step (a) are selected from the group consisting of colloidal silicon dioxide, starch and talc.

14: The process according to claim 5, wherein the lubricants of step (a) are selected from the group consisting of calcium stearate hydrogenated, vegetable oils, mineral oil, polyethylene glycols, stearic acid and sodium stearyl fumarat.

15: A MUPS tablet obtained by the process according to claim 5.
